(19) United States
(12) Patent Application Publication
(10) Pub. No.: US 2020/0402639 A1
(43) Pub. Date: Dec. 24, 2020

(54) SYSTEMS AND METHODS FOR PLANNING AND PERFORMING A COMPUTER ASSISTED PROCEDURE

(71) Applicant: INTELLIJOINT SURGICAL INC., Kitchener (CA)

(72) Inventors: ANDRE NOVOMIR HLADIO, WATERLOO (CA); JOSEPH ARTHUR SCHIPPER, KITCHENER (CA); JUSTIN AARON MICHAEL, VANCOUVER (CA)

(21) Appl. No.: 16/904,907

(22) Filed: Jun. 18, 2020

Related U.S. Application Data
(60) Provisional application No. 62/864,635, filed on Jun. 21, 2019.

(51) Int. Cl. G16H 20/40; A61B 34/10; G16H 50/50; G16H 50/20; G16H 30/40; G16H 40/20; G06Q 10/06; G06F 30/10; G06T 17/00; G06T 3/00

(52) U.S. Cl. CPC G16H 20/40; A61B 34/10; G16H 50/50; G16H 50/20; G16H 30/40; A61B 2090/364; G06Q 10/06316; G06F 30/10; G06T 17/00; G06T 3/0068; A61B 2034/105; G16H 40/20

(57) ABSTRACT

Preoperative planning of a procedure such as an orthopedic procedure is enabled to use first images of patient anatomy in a functional position to define a 3D model and a plan for the procedure and to export the plan to systems configured to use surface information derived from second images of the patient. First images may comprise X-rays including biplanar X-rays. Second images may comprise volumetric datasets such as CT scans. The plans and the surface information may be used together via relative to a common coordinate system. Systems configured to use surface information derived from CT scans, typically of patients in non-functional positions are thus enabled to use plans prepared from images of patients in functional positions such as from (synchronized) biplanar X-rays of patient anatomy.

### CROSS-REFERENCE

This application claims the benefit of U.S. Provisional Application No. 62/864,635, filed Jun. 21, 2019, the entire contents of which are incorporated herein by reference.

### FIELD

The following relates to computing devices and computing methods for planning and performing computer assisted procedures on a patient anatomy.

### BACKGROUND

Various systems and methods are known to plan and then perform a computer assisted procedure such as an orthopaedic procedure on an anatomy of a patient. Preplanning images of the patient may be obtained usually in the form of a computed tomography (CT) scan or other volumetric modality. Further imaging modalities for patients may include X-rays, including synchronized biplanar X-rays. X-ray machines may capture patients in functional positions (e.g. weight bearing positions) such as standing, sitting, etc.), whereas CT or other scanners typically capture patients in non-functional positions (e.g. a supine patient position such as laying on the back.).

Respective 3D models may be produced from the patient images according to the modalities using respective planning systems configured with respective planning software. The respective 3D models (or portions thereof) providing surface information regarding the anatomy and the plans may be communicated to systems to assist with a procedure. Such systems may be for use during the procedure in an operating room or to produce patient specific instruments for use during a procedure. A planning system that produces a 3D model and plan from a volumetric data set is not configured to produce a 3D model and plan from synchronized biplanar X-rays and vice versa.

It may be desired to utilize a plan obtained from patient images capturing a functional position together with surface information derived from patient images in a non-functional position (e.g. from a volumetric dataset) in a computing system to assist with the procedure.

### SUMMARY

Preoperative planning of a procedure such as an orthopedic procedure is enabled to use first images of patient anatomy in a functional position to define a 3D model and a plan for the procedure and to export the plan to systems configured to use surface information derived from second images of the patient. First images may comprise X-rays including biplanar X-rays. Second images may comprise volumetric datasets such as CT scans. The plans and the surface information may be used together via relative to a common coordinate system. Systems configured to use surface information derived from CT scans, typically of patients in non-functional positions are thus enabled to use plans prepared from images of patients in functional positions such as from (synchronized) biplanar X-rays of patient anatomy.

There is provided a method comprising: defining a 3D model from first images of a patient's anatomy; defining a plan using the 3D model to assist to perform a procedure on the patient's anatomy; co-registering the plan and surface information obtained from second images of the patient's anatomy to a common coordinate system; and exporting the plan and the surface information as co-registered for use by a second computing device configured to assist with the procedure.

There is provided a method comprising: co-registering a plan for a procedure with respect to a patient's anatomy with surface information for the patient's anatomy relative to a common coordinate system, the plan defined from first images of the patient's anatomy and the surface information obtained from second images of the patient's anatomy; and providing the plan and the surface information as co-registered for use by a second computing device configured to assist with the procedure.

There is provided a method comprising: receiving surface information for a patient's anatomy; receiving a plan for a procedure for use with the surface information; and using the surface information and the plan to assist to perform the procedure; wherein: the plan and the surface information are co-registered in respect of a common coordinate system; the plan comprises planning data defined to assist to perform the procedure on the patient's anatomy from a 3D model constructed from first images of the patient's anatomy; and the surface information is constructed from second images of the patient's anatomy.

There is provided a computing device comprising a processor and a non-transient storage device storing instructions which when executed by the processor configure the computing device to: define a 3D model from first images of a patient's anatomy; define a plan using the 3D model to assist to perform a procedure on the patient's anatomy; co-register the plan and surface information obtained from second images of the patient's anatomy to a common coordinate system; and export the plan and the surface information as co-registered for use by a second computing device configured to assist with the procedure.

To co-register may comprise defining co-registration information to enable use of the plan and the surface information obtained from second images of the patient's anatomy relative to the common coordinate system. Defining co-registration information may comprise computing a rigid transformation that minimizes a metric of agreement between surface information derived from the first images and the surface information derived from the second images. The computing device may be configured to use an iterative closet point algorithm to minimize the metric of agreement.

Defining co-registration information may comprise receiving a candidate co-registration and providing for display a visualization, metric, or other indicator of agreement between the surface information under the candidate co-registration and the first images alongside a user interface that enables a user to change and/or accept the candidate co-registration

To export may include exporting the co-registration information.

The patient's anatomy in the first images may be in a functional position.

The first images may comprise biplanar X-rays. The biplanar X-rays may comprise synchronized biplanar X-rays.

The patient's anatomy of the second images may be in a non-functional position.

The second images may comprise images defined in accordance with computed tomography (CT) techniques. The second images may comprise DICOM images. The second images comprise a segmented 3D model.

The computing device may be further configured to at least one of: receive the surface information defined from the second images; and, receive the second images and define the surface information from the second images.

The computing device may be further configured to confirm the patient's anatomy of the second images is the same as the patient's anatomy of the first images.

The computing device may be, further configured to confirm the patient's anatomy relates to the correct patient using patient identity data.

The computing device may be further configured to provide workflow to guide a user to provide input to perform at least one of: defining the 3D model; defining the plan; defining the co-registration information; and exporting the plan and surface information.

The computing device may be further configured to determine whether the second images are available and define the co-registration information accordingly.

The computing device may be further configured to define the co-registration information to additionally co-register one or both of the first images and the 3D model for use with the common coordinate system and wherein to export further includes exporting one or both of the first images and the 3D model in the common coordinate system.

These and other aspects will be apparent to a person of skill in the art, including computer readable medium aspects where a non-transient storage device such as a memory or other storage product stores instructions which when executed by a processor of a computing device configure the computing device to perform any of the method aspects herein.

### BRIEF DESCRIPTION OF THE DRAWINGS

FIG. 1 is a block diagram of a computing network including computing devices to perform planning and computing devices to assist with a procedure utilizing a plan where at least one planning system is configured to define a 3D model and plan derived from synchronized biplanar X-rays for use by a computing device to assist with a procedure configured to utilize a 3D model and plan derived from a volumetric dataset.

FIGS. 2-6 are flowcharts of operations for computing device(s) in accordance with examples herein.

The present concept of the invention is best described through certain embodiments thereof, which are described herein with reference to the accompanying drawings, wherein like reference numerals refer to like features throughout. It is to be understood that the term invention, when used herein, is intended to connote the concept underlying the embodiments described below and not merely the embodiments themselves. It is to be understood further that the general concept is not limited to the illustrative embodiments described below and the following descriptions should be read in such light. More than one concept may be shown and described and each may standalone or be combined with one or more others unless stated otherwise.

### DETAILED DESCRIPTION

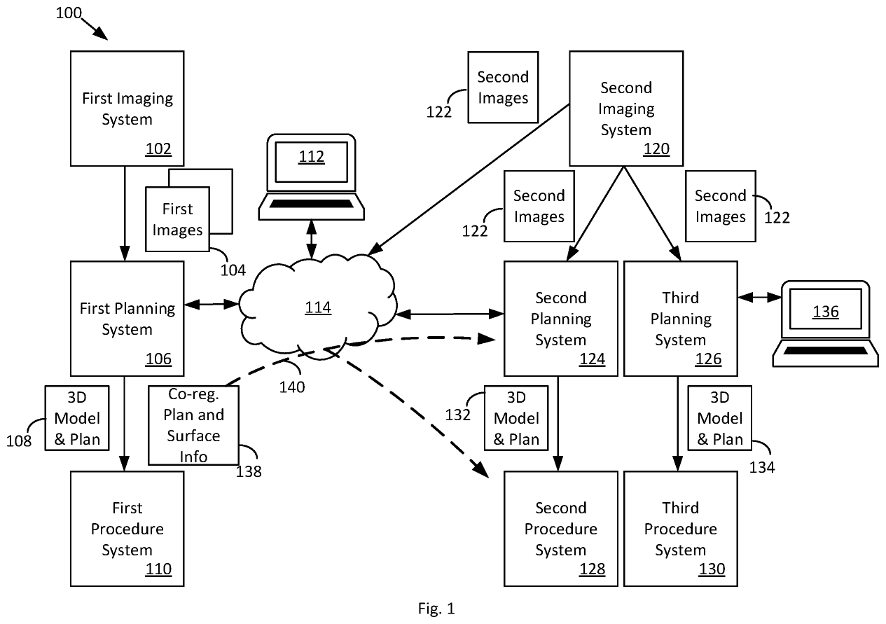

FIG. 1 is a block diagram of a computer network 100 shown in a simplified manner. Network 100 comprises a first imaging system 102 for producing first images 104 of a patient. First imaging system 102 is configured to produce first images 104 in a first modality such as a form of X-ray. First imaging system 102 may comprise an EOS™ X-ray imaging system from EOS imaging to produce biplanar X-rays simultaneously. The patient may be imaged in a functional position. For example in an upright, physiological load-bearing position, in a seated or standing position.

The simultaneous production of LAT (lateral) and AP (anteriorposterior) (or PA) images that are spatially calibrated enables a 3D surface reconstruction of the patient anatomy (skeletal system) such as using appropriate software tools. Other imaging modalities and methods may be used to enable 3D surface reconstruction of a patient in a functional position (for example, conventional x-rays may be inputted into a statistical shape model algorithm to estimate a 3D model of the patient's anatomy).

First images 104 may be communicated to first planning system 106 configured with first planning software (not shown) comprising instructions which when executed configure the operation of the first planning system. First planning system 106 may be configured to define a 3D surface reconstruction (e.g. a 3D model) from the biplanar X-ray images and perform planning to define a plan for an orthopedic procedure such as a spinal, hip or knee procedure. Output from first planning system 106 may comprise 3D model and plan 108. First planning system 106 may be enabled to produce surface 3D reconstructions with visual and quantitative parametric analysis of the skeletal system in a normal upright position. Most patient imaging modalities, including computerized tomography (CT) scanning, that enable 3D volumetric reconstructions require a patient to be in a supine position rather than in a more clinically desirable load bearing position (e.g. a functional position).

First planning system 106 may be enabled to (automatically) calculate and record relevant orthopaedic clinical parameters such as to assist with planning.

3D model and plan 108 may be communicated to a first procedure system 110. First procedure system 110 may comprise a computing system to assist with the performance of the procedure in accordance with the plan. First procedure system 110 may comprise or be in communication with a localization system (e.g. a surgical navigation or robotic system) generating data to track objects during an orthopedic procedure. The objects may be surgical tools or bones, etc. of the patient's anatomy. First procedure system 110 may comprise software (instructions) to configure operation of the first procedure system 110. First procedure system 110 may present workflow via graphical or other user interfaces. Workflow may guide a registration of the 3D space of a camera of the localization system with a coordinate system of the first procedure system and a coordinate system of the 3D model. The 3D model may be visualized. Tracked objects may be co-displayed with the 3D model. Patient measurements may be taken using the localization system and workflow followed such as to perform the procedure. The procedure may be a hip replacement, for example, performing placement of one or more implants. The plan (108) may comprise plan data such as target measurements. For example, with respect to total hip arthroplasty, the plan may comprise one or more of: acetabular inclination; acetabular anteversion; femoral offset; femoral anteversion; and, hip center of rotation.

First planning system 106 may be enabled to communicate with a remote computing device 112 such as a laptop or other computer having a different form factor to perform planning. First planning system 106 may be cloud or web enabled and communicate via network 114 such as a public network, albeit, in a secure manner.

Computer network 100 further comprises a second imaging system 120 producing second images 122 (three instances are shown). Second imaging system is configured to produce images in a second modality such as a CT scan so that a patient may be imaged to produce both first images 104 and second images 122. Second images 122 are typically of a patient in a non-load bearing (or differently load bearing) position such as supine position compared to the position of the first images 104. Second images 122 (and first images 104) may be produced or converted to a DICOM (Digital Imaging and Communications in Medicine) format. Second images 122 may be segmented to generate a segmented 3D model.

Computer network 100 further comprises, respectively, a second planning system 124 and third planning system 126, a second procedure system 128 and a third procedure system 130.

Second images 122 may be communicated to second planning system 124 configured with second planning software (not shown) comprising instructions which when executed configure the operation of the second planning system 124. Conventionally, second planning system 124 may be configured to define a 3D reconstruction comprising surface information (e.g. a 3D model) from second images 122. Second planning system 124 may be enabled to perform planning to define a plan for an orthopedic procedure such as a spinal, hip or knee procedure, using the 3D model from second images 122. Conventionally, output from second planning system 124 may comprise 3D model and plan 132 derived from the second images. In accordance with an example herein, second planning system may be adapted to additionally receive a 3D model and plan from first planning system 106 as further described.

3D model and plan 132 is communicated to second procedure system 128. In one example, second procedure system 128 is configured to produce patient specific instruments for use during a patient procedure. Patient specific instruments are typically 3D printed to specifications for the patient's anatomy. Though not shown, a further procedure system may be provided to assist in an operating room during the procedure to deliver the procedure using the patient specific instruments and in accordance with the plan (e.g. 132). The further procedure system may be configured similarly to first procedure system 110.

Second images 122 may be communicated to third planning system 126 configured with third planning software (not shown) comprising instructions which when executed configure the operation of the third planning system 126. Conventionally, third planning system 126 may be configured to define a 3D reconstruction comprising surface information (e.g. a 3D model) from second images 122. Third planning system 126 may be enabled to perform planning to define a plan for an orthopedic procedure such as a spinal, hip or knee procedure using the 3D model from second images 122. Conventionally, output from third planning system 126 may comprise a 3D model and plan 134 derived from the second images 122. In accordance with an example herein, third planning system 126 may be adapted to additionally receive a 3D model and plan from first planning system 106 as further described.

3D model and plan 134 is communicated to third procedure system 130. In one example, third procedure system 130 is configured as a robotic platform to provide robotic assistance to a surgeon to perform an orthopaedic procedure on the patient in accordance with the plan (e.g. 134). Third procedure system 130 may be configured similarly to first procedure system 110 but with control and input for robotic components.

Either or both of second planning system 124 and third planning system 126 may be enabled with cloud or web-based interfaces such as to work with a remote computing device 112 or 136. Remote device 136 is shown coupled to third planning system 126 via a local connection such as a local area network merely as an example. Each of the planning systems has an associated data store (e.g. database or other storage) to store patient data, first and second images, 3D models, planning data, etc.

It will be understood that a 3D model (e.g. 3D surface reconstruction) produced from biplanar X-rays is produced using various modelling and statistical techniques and may represent less accurate patient information than surface information (e.g. a 3D model/3D volumetric reconstruction) produced from a CT scan. Surface information for the patient is typically used in assisting with the procedure such as to register the patient's anatomy to a localization system coupled to a computing device or to produce patient specific instruments (e.g. jigs).

It will be understood that a plan produced from images of the patient in a functional position such as an active weight bearing position (e.g. standing and/or seated) may be more clinically relevant than a plan produced from images of the patient in a non-functional position (e.g. a supine position where joints of interest are not bearing weight or not bearing sufficient weight).

It will be understood that the respective first images 104 and second images 122 as well as the respective 3D models defined therefrom and any plan have or are defined relative to respective coordinate systems.

Thus, it may be desired to enable the use of a plan defined from first images for use with surface information defined from second images to assist to perform a procedure.

In one example, a planning system, such as first planning system 106, may be configured to receive second images 122 (e.g. a CT scan in DICOM or segmented form) for use in operations to relate a plan to surface information derived from the second images.

As previously noted, first planning system 106 may be enabled to:
receive the first images such as biplanar X-ray images (e.g. AP and LAT images of first images 104),
define (e.g. construct) a 3D model therefrom; and
define a plan for a procedure (e.g. in response to user input) in accordance with the 3D model.

First planning system 106 may also be enabled to:
Co-register the plan obtained from first images of the patient's anatomy and surface information obtained from second images of the patient's anatomy to a common coordinate system. Co-registration here may comprise defining one or both of the plan and the surface information in the common coordinate system or may comprise defining co-registration information for application to one or both of the plan and the surface information to transform to the common coordinate system; and
export the plan and the surface information in the common coordinate system, which may include exporting co-registration information to perform a transformation, for use by a second computing device configured to assist with the procedure.

First planning system 106 may also be enabled to receive the surface information already derived from the second images or receive the second images and define the surface information. It will be understood that the surface information derived from the second images may comprise a 3D model.

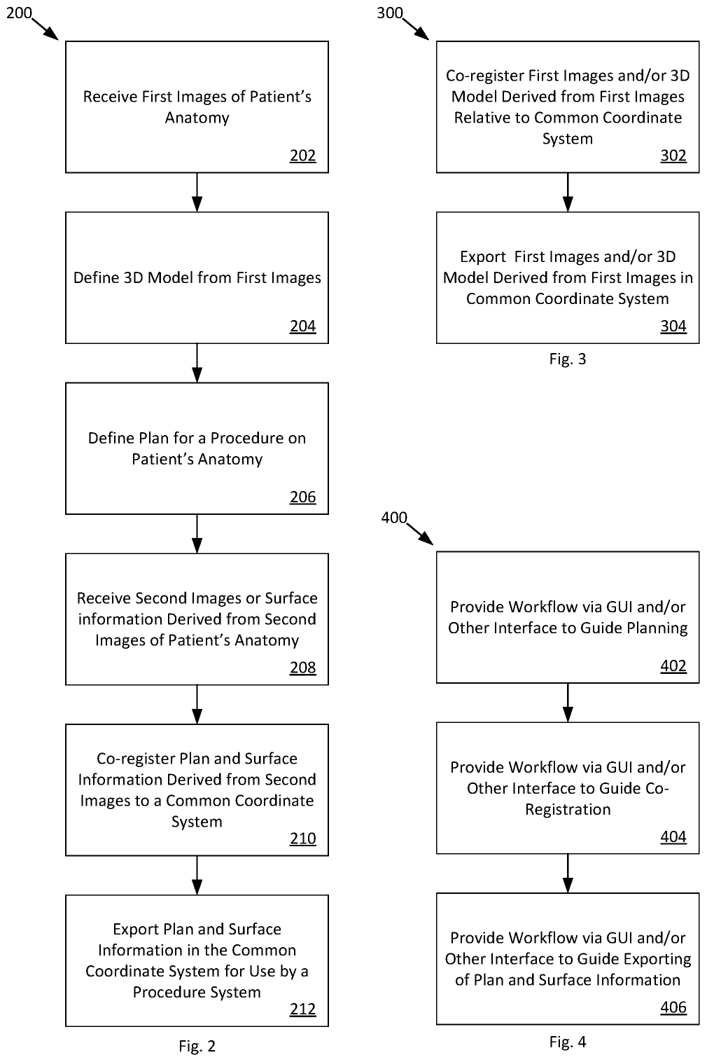

FIG. 2 is a flowchart of operations 200 comprising steps 202, 204, 206, 208, 210 and 212 according to an example for first planning system 106.

The patient's anatomy in the first images may be in a functional position. The first images may comprise biplanar X-rays. The biplanar X-rays may comprise synchronized biplanar X-rays.

The patient's anatomy of the second images may be in a non-functional position. The second images may comprise images defined in accordance with computed tomography (CT) techniques. second images may comprise DICOM images. The second images may comprise a segmented 3D model.

It is understood that the various data comprising the first images, 3D models constructed from the first images, plans, second images, and surface information constructed from the second images may be defined using respective coordinate systems. A coordinate system for the first images, 3D model and plan may be the same coordinate system because the 3D model and plan are derived from the first images (e.g. are first image related data). Similarly a coordinate system for the second images and the surface information constructed therefrom may be the same (e.g. as the surface information is second image related data).

To use any of the first images and/or first image related data together with any of the second images or second image related data, and more particularly, to use the plan with the surface information, a common coordinate system may be employed. This common coordinate system could be either one of the respective coordinates systems or a third coordinate system. Defining co-registration information may thus comprise defining transforms or other operative data to make the plan useful with the coordinate system of the surface information or vice versa or to make the plan and surface information useful with a third coordinate system. The plan and surface information may be co-registered (e.g. expressed) relative to a common coordinate system.

FIG. 3 shows a flow chart of operations 300 according to an example for first planning system 106 where it is enabled to co-register one or both of the first images and the 3D model derived from the first images relative to the common coordinate system (302) and to export one or both of the first images and the 3D model derived from the first images accordingly (304).

FIG. 4 shows a flow chart of operations 400 according to an example for first planning system 106. First planning system 106 may be enabled to provide workflow including a graphical user interface (GUI) and/or other interface, to guide the planning (402). First planning system 106 may be enabled to provide workflow including a graphical user interface (GUI) and/or other interface to guide co-registration (404). First planning system 106 may be enabled to provide workflow including a graphical user interface (GUI) and/or other interface to guide exporting (406).

First planning system 106 may guide requesting and/or receiving of the first images 104 and second images 122, to produce a 3D model defined from the first images and to define a plan using that 3D model. The workflow may be configured to be responsive to the type of procedure to be performed. Options may be provided to configure the plan such as to select a class (or classes) of implants, etc. Some clinical measurements of patient anatomy may be automatically determined. First planning system 106 may be enabled to display first images 104 and/or the 3D model and receive user input to mark locations relative to the 3D model (and/or first images) and to initiate calculations, etc.

Implant parameters may be calculated by first planning system 106, to aid in automatic and/or user-based planning. Implant parameters may include a metric of how good a current implant's (or set of implants') position is. For example, risk of impingement during a functional range of motion may be an implant parameter that could be calculated (and displayed) to aid in planning. This parameter may be updated in real time, responsive to a user changing the plan. In another example, an implant parameter is a metric of how appropriately aligned an implant is with respect to a weight bearing axis, optionally in more than one functional position.

Should a CT scan (i.e. second images 122) not be available to first planning system 106 during a planning session, some operations may not be permitted. For example co-registration to the CT scan would not be permitted. Controls therefor (e.g. to invoke/perform the operation) may be greyed out or not displayed.

First planning system 106 may be enabled with controls to invoke the output of data (e.g. to save to a data store and/or export such as to communicate to another computing system). The co-registered plan and surface information 138 may be communicated (e.g. via network 114 or otherwise) to another computing device. Dotted line arrow 140 represents a network communication to any of second planning system 124, third planning system 126, second procedure system 128 and third procedure system 130 as an example. Co-registered plan and surface information 138 may be received by any of such computing devices and shared with others (not shown).

First planning system 106 may enable exporting of the plan and surface information to various procedure systems. Planning system 106 may implement a user interface that enables a user to select the procedure system they would like to export to. The user interface may be adaptive to reflect the available procedure system options. For example, the user interface may grey out or not show options that are unavailable. Availability of procedure system options may be determined by planning system 106 based on a number of parameters accessible in memory or over the network, including: adequate lead time for patient specific instrument manufacturing; imaging modality and parameters (e.g. resolution, intensity, slice thickness); availability of the particular procedure system for a particular surgeon and/or hospital; surgeon preferences; status of surgeon plan approval (some systems may require a surgeon to approve a plan before allowing data to be exported); surgical procedure type and approach; patient preferences; etc.

In another data transfer example, a 3D model and plan, such as co-registered plan and surface information 138, may be communicated in an encoded form such as encoding data in a quick response (QR) code or other matrix barcode. Such may be stored electronically and/or printed and presented to optically transfer the information. In addition to anatomical measurements etc., patient identifying data may be transferred. Such data is typically available to a planning system, for example, associated with planning data and/or image data for a patient. Confirmation of patient identity (e.g. that the image data, 3D model and plan etc. relate to the correct patient) may be performed using such data.

Workflow or other operations of first planning system 106 may confirm that the patient of the first images 104 and the patient of the second images 122 are the same patient. For example, first planning system 106 may be enabled to compare patient anatomy derived from the respective first images and second images to ensure there is a sufficient match.

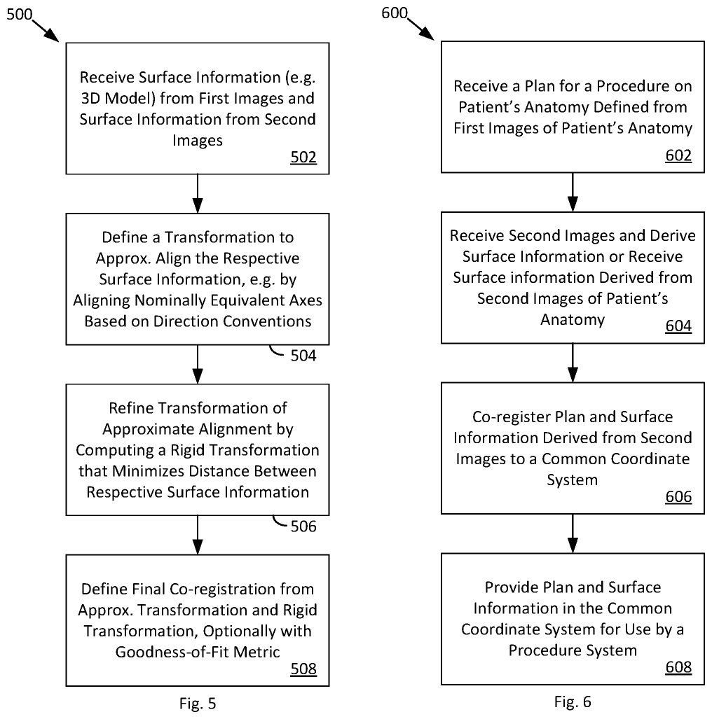

FIG. 5 shows a flow chart of operations 500 according to an example for co-registering the surface information derived from the first and second images, respectively, of the patient's anatomy. At 502 the computing device receives the surface information from the first and second images of patient's anatomy, respectively. At 504 a transformation is defined that approximately aligns the two sets of surface information, such as by aligning nominally equivalent axes based on a priori direction conventions.

At 506 the approximate alignment from 504 is refined by computing a rigid transformation that minimizes the distance between all or a subset of the two sets of surface information (e.g. pelvis only) in the least squares sense. Least squares optimization may be performed using the iterative closest point algorithm or through a more generalized optimization scheme. At 508 the approximate alignment and refined registration are concatenated to define the final co-registration between the two sets of surface information, optionally with a goodness-of-fit metric describing the agreement between the two surfaces after alignment.

Alternatively to the example operations described in FIG. 5, co-registration may co-register the surface information derived from the second images to the first images directly, omitting the need for surface information derived from the first images. An example of such a co-registration process would be to produce a co-registration transform by computing a numeric metric of agreement between the first images and a candidate transform of the surface information, and use a computer implemented method to optimize that metric as a function of the transform (e.g. as a minimization problem to minimize a distance measure). The metric of agreement could be calculated based on the first images themselves (e.g. image similarity metrics between actual and simulated biplanar x-ray images) or based on information derived from the first images (e.g. similarity metrics between 2D segmentations in biplanar x-rays and boundaries in simulated projections). Alternatively to computerized optimization, a numeric metric, visualization or other indicator may be computed and displayed alongside a user interface that enables a user to change and/or accept the candidate co-registration.

FIG. 6 is a flowchart of operation 600 for a computing device such as one of second planning system 124 or third planning system 126 according to an example. In the present example, the computing device that defines the plan need not be the same computing device that co-registers the plan and the surface information.

At 602 the computing device receives a plan for a procedure on a patient's anatomy defined from first images of the anatomy (e.g. from a 3D model constructed therefrom). At 604 second images of the patient's anatomy are received and surface information is derived therefrom or surface information from the second images is received. Note tht the computing device may be enabled to do one of receiving second images and receiving surface information. In need not be enabled to do both of such operations.

At 606 operations co-register the plan and surface information (derived from the second images) to a common coordinate system for use to assist with the procedure.

At 608, operations provide the plan and surface information in the common coordinate system for use by a procedure system. To provide in this context may comprise exporting/communicating to another computing device or making available the plan and surface information to operations on a same computing device that performed the co-registration.

In an example of the process described in FIG. 6, the computing device may receive a plan and surface information corresponding to biplanar x-ray images (first images) and surface information corresponding to a CT image (second images) and co-register the two sets of surface information. The computing device may then transmit the plan derived from the biplanar x-ray images and the surface information corresponding to the CT image expressed in the coordinate system of the plan (an example of a common coordinate system).

Alternatively, rather than transmitting the plan and surface information where both are finally expressed in a common coordinate system, the computing device may instead transmit the plan and surface information in disparate coordinate systems along with the co-registration information necessary to transform one or both such that they are expressible in a common coordinate system.

Each planning system and procedure system herein comprises a computing device having or coupled to a display and input device(s) such as a keyboard. Other input devices may include a microphone, pointing device, touchscreen, etc. (not shown). Other output devices not shown may include a speaker, lights, etc. Each planning system and procedure system may be configured such as via instructions (software) stored in a storage device (e.g. a memory or other non-transitory medium) for execution by a processor. As noted, for the planning systems, the instructions may provide a graphical user interface to present image data and receive input such as to define anatomical measurements relative to the image data. Planning systems may be configured via workflow (from the software instructions) to guide a user to provide input for the operations of the respective planning system. Similar considerations apply to the respective procedure systems viz. a vis instructions may provide a graphical user interface to present image data (e.g. 3D model data), to receive and present live data such as tracking data from a localization system and receive input such as to define anatomical measurements relative to the image data, etc. Procedure systems may be configured via workflow (from the software instructions) to guide a user to provide input for the operations of the respective procedure system and may receive input from localization systems or other coupled systems during operations to trigger or gate certain workflow. Planning data may be presented. Warnings and other guidance may be provided responsive to the planning data and/or tracking data or other configuration data, etc.

Practical implementation may include any or all of the features described herein. These and other aspects, features and various combinations may be expressed as methods, apparatus, systems, means for performing functions, program products, and in other ways, combining the features described herein. A number of embodiments have been described. Nevertheless, it will be understood that various modifications can be made without departing from the spirit and scope of the processes and techniques described herein. Accordingly, other embodiments are within the scope of the following claims.

Throughout the description and claims of this specification, the word “comprise” and “contain” and variations of them mean “including but not limited to” and they are not intended to (and do not) exclude other components, integers or steps. Throughout this specification, the singular encompasses the plural unless the context requires otherwise. In particular, where the indefinite article is used, the specification is to be understood as contemplating plurality as well as singularity, unless the context requires otherwise. Herein, “A and/or B” means A or B or both A and B.

Features, integers, characteristics, etc. described in conjunction with a particular aspect, embodiment or example are to be understood to be applicable to any other aspect, embodiment or example unless incompatible therewith. All of the features disclosed herein (including any accompanying claims, abstract and drawings), and/or all of the steps of any method or process so disclosed, may be combined in any combination, except combinations where at least some of such features and/or steps are mutually exclusive. The invention is not restricted to the details of any foregoing examples or embodiments. The invention extends to any novel one, or any novel combination, of the features disclosed in this specification (including any accompanying claims, abstract and drawings).

What is claimed is:

1. A computing device comprising a processor and a non-transient storage device storing instructions which when executed by the processor configure the computing device to:
define a 3D model from first images of a patient's anatomy;
define a plan using the 3D model to assist to perform a procedure on the patient's anatomy;
co-register the plan and surface information obtained from second images of the patient's anatomy to a common coordinate system; and
export the plan and the surface information as co-registered for use by a second computing device configured to assist with the procedure.

2. The computing device of claim 1, wherein to co-register comprises defining co-registration information to enable use of the plan and the surface information obtained from second images of the patient's anatomy relative to the common coordinate system; and wherein defining co-registration information comprises computing a rigid transformation that minimizes a metric of agreement between surface information derived from the first images and the surface information derived from the second images.

3. The computing device of claim 2, configured to use an iterative closet point algorithm to minimize the metric of agreement.

4. The computing device of claim 2, wherein defining co-registration information comprises receiving a candidate co-registration and providing for display a visualization, metric, or other indicator of agreement between the surface information under the candidate co-registration and the first images alongside a user interface that enables a user to change and/or accept the candidate co-registration

5. The computing device of claim 2, wherein to export includes exporting the co-registration information.

6. The computing device of claim 1, wherein the patient's anatomy in the first images is in a functional position.

7. The computing device of claim 1, wherein the first images comprise biplanar X-rays.

8. The computing device of claim 1, wherein the patient's anatomy of the second images is in a non-functional position.

9. The computing device claim 1, wherein the second images comprise at least one of:
images defined in accordance with computed tomography (CT) techniques;
DICOM images; and
a segmented 3D model.

10. The computing device of claim 1, further configured to at least one of: receive the surface information defined from the second images; and, receive the second images and define the surface information from the second images.

11. The computing device claim 1, further configured to provide workflow to guide a user to provide input to perform at least one of:
defining the 3D model;
defining the plan;
defining the co-registration information; and
exporting the plan and surface information.

12. The computing device of claim 1, further configured to define the co-registration information to additionally co-register one or both of the first images and the 3D model for use with the common coordinate system and wherein to export further includes exporting one or both of the first images and the 3D model in the common coordinate system.

13. A computing device comprising a processor and a non-transient storage device storing instructions which when executed by the processor configure the computing device to:
co-register a plan for a procedure with respect to a patient's anatomy with surface information for the patient's anatomy relative to a common coordinate system, the plan defined from first images of the patient's anatomy and the surface information obtained from second images of the patient's anatomy; and
export the plan and the surface information as co-registered for use by a second computing device configured to assist with the procedure.

14. The computing device of claim 13, wherein to co-register comprises defining co-registration information to enable use of the plan with the surface information obtained from the second images of the patient's anatomy relative to the common coordinate system; and wherein defining co-registration information comprises computing a rigid transformation that minimizes a metric of agreement between surface information derived from the first images and the surface information derived from the second images.

15. The computing device of claim 14, wherein defining co-registration information comprises receiving a candidate co-registration and providing for display a visualization, metric, or other indicator of agreement between the surface information under the candidate co-registration and the first images alongside a user interface that enables a user to change and/or accept the candidate co-registration.

16. The computing device of claim 14, wherein to export includes exporting the co-registration information.

17. The computing device of claim 13, wherein the patient's anatomy in the first images is in a functional position and wherein the patient's anatomy of the second images is in a non-functional position.

18. The computing device of claim 13, wherein the first images comprise biplanar X-rays.

19. The computing device of claim 13, wherein the second images comprise images defined in accordance with computed tomography (CT) techniques.

20. The computing device of claim 13, wherein the computing device is at least one of: further configured to receive the plan defined from the first images; and further configured to receive the second images and define the surface information from the second images.
